(19) United States
(12) Patent Application Publication
(10) Pub. No.: US 2009/0048308 A1
(43) Pub. Date: Feb. 19, 2009

(54) VLA-4 Antagonists

(76) Inventors: Nicholas S. Stock, San Diego, CA (US); Nicholas D. Smith, San Diego, CA (US); Benito Munoz, San Diego, CA (US)

Correspondence Address: MERCK AND CO., INC, P O BOX 2000, RAHWAY, NJ, 07065-0907, US

(21) Appl. No.: 11/918,100

(22) PCT Filed: Apr. 10, 2006
(86) PCT No.: PCT/US2006/013254

§ 371 Date: Oct. 9, 2007

Related U.S. Application Data
(60) Provisional application No. 60/671,352, filed on Apr. 14, 2005.

(51) Int. Cl. A61K 31/4439; C07D 401/12; A61P 35/02; A61P 9/10; A61P 35/00; A61P 11/06; A61P 19/02; A61P 1/00

(52) U.S. Cl. 514/343; 546/278.7

(57) ABSTRACT

4-Thio, 4-sulfinyl and 4-sulfonyl proline derivatives of the present invention are antagonists of the VLA-4 integrin and are useful in the treatment, prevention and suppression of diseases mediated by VLA-4-binding and cell adhesion and activation. Moreover, the compounds of the present invention demonstrate significant receptor occupancy of VLA-4 bearing cells after oral administration and are suitable for once-, twice-, or thrice-a-day oral administration. This invention also relates to compositions containing such compounds and methods of treatment using such compounds.

### BACKGROUND OF THE INVENTION

VLA-4 (“very late antigen-4”; CD49d/CD29; or α₄β₁) is an integrin expressed on all leukocytes, except platelets and mature neutrophils, including dendritic cells and macrophage-like cells and is a key mediator of the cell-cell and cell-matrix interactions of these cell types. The ligands for VLA-4 include vascular cell adhesion molecule-1 (VCAM-1), the CS-1 domain of fibronectin (FN), and the matrix protein, osteopontin. Neutralizing anti-α₄ antibodies or blocking peptides that inhibit the interaction between VLA-4 and its ligands have been shown to be efficacious both prophylactically and therapeutically in several animal models of disease including asthma, multiple sclerosis, inflammatory bowel disease, multiple myeloma, and rheumatoid arthritis.

The humanized monoclonal antibody against α₄, natalizumab (Tysabri®, Elan/Biogen), has demonstrated efficacy in the treatment of multiple sclerosis (D. H. Miller et al., New England Journal of Medicine, 348, 15 (2003)) and Crohn's disease (S. Ghosh et al. New England Journal of Medicine, 348, 23 (2003)). There are also several VLA-4 antagonists in early clinical trials for treatment of asthma, arthritis, multiple sclerosis, and Crohn's disease.

In the early clinical trials with natalizumab, lymphocytosis (a surrogate marker for blockade of VLA-4 function) and >80% receptor occupancy were observed. A small molecule VLA-4 antagonist was reported to demonstrate functional activity in the rat experimental autoimmune encephalomyelitis (EAE) assay, an animal model of multiple sclerosis following subcutaneous administration (D. R. Leone et al., J. Pharmacol. Exper. Therap., 305, 1150 (2003). This compound was shown to induce lymphocytosis, and to have a slow dissociation rate (off-rate) resulting in significant and sustained receptor occupancy on VLA-4-bearing cells. There was a positive correlation between receptor occupancy, lymphocytosis, and efficacy in the EAE model described in this manuscript.

A series of isonicotinoyl-L-aminophenylalanine derivatives shown to possess slow dissociation (off-rate) from VLA-4 on Jurkat cells were reported in G. Doherty et al., Bioorganic & Medicinal Chemistry Letters, 13, 1891 (2003). However, the compound that was further characterized demonstrated very poor pharmacokinetic properties such as low oral bioavailability, moderate to high plasma clearance and a short half-life rendering it unsuitable for oral administration. Compounds of the present invention are potent antagonists of VLA-4 capable of achieving and maintaining receptor occupancy for a time sufficient to allow for oral administration.

### SUMMARY OF THE INVENTION

4-thio, 4-sulfinyl and 4-sulfonyl proline derivatives of the present invention are antagonists of the VLA-4 integrin and are useful in the treatment, prevention and suppression of diseases mediated by VLA-4-binding and cell adhesion and activation. Moreover, the compounds of the present invention demonstrate significant receptor occupancy of VLA-4 bearing cells after oral administration and are suitable for once-, twice-, or thrice-a-day oral administration. This invention also relates to compositions containing such compounds and methods of treatment using such compounds.

### DETAILED DESCRIPTION OF THE INVENTION

The present invention encompasses a genus of compounds of Formula I:

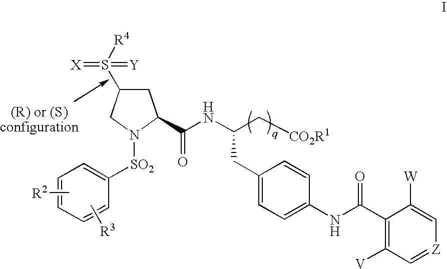

or a pharmaceutically acceptable salt thereof, wherein:
q is 0 or 1;
V and W are independently chosen from (1) C₁₋₃alkyl, (2) halogen, and (3) C₁₋₃alkoxy;
X and Y may independently be oxygen or not present;

### Z is N or N+O—;

R¹ is selected from (1) hydrogen, (2) C₁₋₁₀alkyl, (3)-(C₁₋₁₀alkyl)-aryl, (4) —(C₁₋₁₀alkyl)-O—C₁₋₁₀alkyl, (5) —(C₁₋₁₀ alkyl)-OC(O)—C₁₋₁₀ alkyl, (6) —(C₁₋₁₀alkyl)-OC(O)-aryl, (7) —(C₁₋₁₀ alkyl)-OC(O)O—C₁₋₁₀alkyl, and (8) —(C₁₋₁₀alkyl)-N⁺(C₁₋₃ alkyl)₃; wherein alkyl is optionally substituted with one to three substituents independently selected from R^{a}, and aryl is optionally substituted with one to three substituents independently selected from R^{b};
R² and R³ are independently selected from H, —SO₂—C₁₋₃ alkyl, CN, CF₃, OCF₃, and halogen;
R⁴ is selected from the group consisting of: C₁₋₁₀ alkyl, C₂₋₁₀alkenyl, C₂₋₁₀alkynyl, Cy and Cy-C₁₋₁₀ alkyl, wherein alkyl, alkenyl, alkynyl and Cy are optionally substituted with one to four substituents independently selected from R^{c};
R^{a} is selected from (1) —OR^{d}, (2) —NR^{d}S(O)ₘR^{e}, (3) —NO₂, (4) halogen, (5) —S(O)ₘR^{d}, (6) —SR^{d}, (7) —S(O)₂OR^{d}, (8) —S(O)ₘNR^{d}R^{e}, (9) —NR^{d}R^{e}, (10) —O(CR^{f}R^{g})ₙNR^{d}R^{e}, (11) —C(O)R^{d}, (12) —CO₂R^{d}, (13) —CO₂(CR^{f}R^{g})ₙCONR^{d}R^{e}, (14) —OC(O)R^{d}, (15) —CN, (16) —C(O)NR^{d}R^{e}, (17) —NR^{d}C(O)R^{e}, (18) —OC(O)NR^{d}R^{e}, (19) —NR^{d}C(O)OR^{e}, (20) —NR^{d}C(O)NR^{d}R^{e}, (21) —CR^{d}(N—OR^{e}), (22) CF₃, (23) —OCF₃, (24) C₃₋₈cycloalkyl, and (25) heterocyclyl; wherein cycloalkyl and heterocyclyl are optionally substituted with one to three groups independently selected from R^{c};
R^{b} is selected from (1) a group selected from R^{a}, (2) C₁₋₁₀ alkyl, (3) C₂₋₁₀ alkenyl (4) C₂₋₁₀ alkynyl, (5) aryl, and (6) —(C₁₋₁₀alkyl)-aryl, wherein alkyl, alkenyl, alkynyl, and aryl are optionally substituted with one to three substituents selected from a group independently selected from R^{c};
R^{c} is (1) halogen, (2) amino, (3) carboxy, (4) C₁₋₄alkyl, (5) C₁₋₄alkoxy, (6) aryl, (7) —(C₁₋₄-alkyl)-aryl, (8) hydroxy, (9) CF₃, (10) OC(O)C₁₋₄alkyl, (11) —CN, and (12) —SO₂C₁₋₁₀alkyl;
R^{d} and R^{e} are independently selected from hydrogen, C₁₋₁₀ alkyl, C₂₋₁₀alkenyl, C₂₋₁₀alkynyl, Cy and Cy-C₁₋₁₀ alkyl, wherein alkyl, alkenyl, alkynyl and Cy are optionally substituted with one to four substituents independently selected from R^{c}; or
R^{d} and R^{e} together with the atom(s) to which they are attached form a heterocyclic ring of 4 to 7 members containing 0-2 additional heteroatoms independently selected from O, S and N—R^{h}; wherein said ring is optionally substituted with one to four substituents independently selected from R^{c}C;
R^{f} and R^{g} are independently selected from hydrogen, C₁₋₁₀alkyl, Cy and Cy-C₁₋₁₀alkyl; or
R^{f} and R^{g} together with the carbon to which they are attached form a ring of 5 to 7 members containing 0-2 heteroatoms independently selected from oxygen, sulfur and nitrogen;
R^{h} is selected from R^{f} and —C(O)R^{f};
Cy is selected from cycloalkyl, heterocyclyl, aryl, and heteroaryl;
each m is independently 0, 1 or 2; and
each n is independently 1, 2, 3, or 4.

Within this genus, the invention encompasses a subgenus of compounds of Formula I wherein one of V and W is halogen and the other is selected from halogen, C₁₋₃alkyl and C₁₋₃ alkoxy. Within this subgenus of compounds, the invention encompasses a class of compounds of Formula I wherein one of V and W is chloro and the other is chloro or methoxy. Within this class, the invention encompasses a subclass of compounds wherein V and W are each chloro.

Also within the genus of compounds, the invention encompasses a subgenus of compounds of Formula I wherein R¹ is selected from the group consisting of: hydrogen, C₁₋₄alkyl, —(C₁₋₄alkyl)OC(O)—C₁₋₄alkyl, and —(C₁₋₄alkyl)OC(O)—C₁₋₄alkyl.

Also within the genus of compounds, the invention encompasses a subgenus of compounds of Formula I wherein R¹ is hydrogen.

Also within the genus of compounds, the invention encompasses a subgenus of compounds of Formula I wherein R¹ is C₁₋₄alkyl.

Also within the genus of compounds, the invention encompasses a subgenus of compounds of Formula I wherein R² is hydrogen and R³ is CN.

Also within the genus of compounds, the invention encompasses a subgenus of compounds of Formula I wherein R⁴ is selected from the group consisting of: C₁₋₆alkyl, cycloalkyl and aryl.

Also within the genus of compounds, the invention encompasses a subgenus of compounds of Formula Ia:

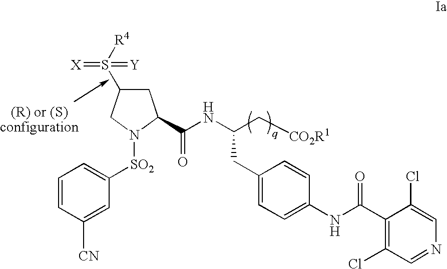

or a pharmaceutically acceptable salt thereof, wherein
q is 0 or 1;
X and Y may independently be oxygen or not present;
R¹ is selected from hydrogen and ethyl; and
R⁴ is selected from C₁₋₆alkyl, cyclopentyl, cyclohexyl and phenyl.

Within this subgenus, the invention encompasses a class of compounds of Formula Ia wherein q is 0.

Also within this subgenus, the invention encompasses a class of compounds of Formula Ia wherein q is 1.

Also within this subgenus, the invention encompasses a class of compounds of Formula Ia wherein X and Y are not present.

Also within this subgenus, the invention encompasses a class of compounds of Formula Ia wherein X is oxygen and Y is not present.

Also within this subgenus, the invention encompasses a class of compounds of Formula Ia wherein X and Y are oxygen.

The invention also encompasses the species described below.

In another aspect the present invention provides a method for the prevention or treatment of diseases, disorders, conditions or symptoms mediated by cell adhesion in a mammal which comprises administering to said mammal an effective amount of a compound of Formula I. This aspect includes the use of a compound of Formula I in the manufacture of a medicament for the treatment of diseases, disorders, conditions or symptoms mediated by cell adhesion in a mammal. In one embodiment said disease or disorder is selected from asthma, allergic rhinitis, chronic obstructory pulmonary disease (COPD), multiple sclerosis, atherosclerosis, inflammatory bowel disease, rheumatoid arthritis, organ transplantation, acute leukemia, and sickle cell anemia.

In another aspect the present invention provides a method for preventing the action of VLA-4 in a mammal which comprises administering to said mammal a therapeutically effective amount of a compound of Formula I.

Another aspect of the present invention provides a pharmaceutical composition which comprises a compound of Formula I and a pharmaceutically acceptable carrier.

“Alkyl”, as well as other groups having the prefix “alk”, such as alkoxy, alkanoyl, means carbon chains which may be linear or branched or combinations thereof. Examples of alkyl groups include methyl, ethyl, propyl, isopropyl, butyl, sec- and tert-butyl, pentyl, hexyl, heptyl, octyl, nonyl, and the like.

“Alkenyl” means carbon chains which contain at least one carbon-carbon double bond, and which may be linear or branched or combinations thereof. Examples of alkenyl include vinyl, allyl, isopropenyl, pentenyl, hexenyl, heptenyl, 1-propenyl, 2-butenyl, 2-methyl-2-butenyl, and the like.

“Alkynyl” means carbon chains which contain at least one carbon-carbon triple bond, and which may be linear or branched or combinations thereof. Examples of alkynyl include ethynyl, propargyl, 3-methyl-1-pentynyl, 2-heptynyl and the like.

“Cycloalkyl” means mono- or bicyclic saturated carbocyclic rings, each of which having from 3 to 10 carbon atoms. The term also includes monocyclic rings fused to an aryl group in which the point of attachment is on the non-aromatic portion. Examples of cycloalkyl include cyclopropyl, cyclobutyl, cyclopentyl, cyclohexyl, cycloheptyl, tetrahydronaphthyl, decahydronaphthyl, indanyl, and the like.

“Aryl” means mono- or bicyclic aromatic rings containing only carbon atoms. The term also includes aryl group fused to a monocyclic cycloalkyl or monocyclic heterocyclyl group in which the point of attachment is on the aromatic portion. Examples of aryl include phenyl, naphthyl, indanyl, indenyl, tetrahydronaphthyl, 2,3-dihydrobenzofuranyl, dihydrobenzopyranyl, 1,4-benzodioxanyl, and the like.

“Heteroaryl” means a mono- or bicyclic aromatic ring containing at least one heteroatom selected from N, O and S, with each ring containing 5 to 6 atoms. Examples of heteroaryl include pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furanyl, triazinyl, thienyl, pyrimidyl, pyridazinyl, pyrazinyl, benzoxazolyl, benzothiazolyl, benzimidazolyl, benzofuranyl, benzothiophenyl, furo(2,3-b)pyridyl, quinolyl, indolyl, isoquinolyl, and the like.

“Heterocyclyl” means mono- or bicyclic saturated rings containing at least one heteroatom selected from N, S and O, each of said ring having from 3 to 10 atoms in which the point of attachment may be carbon or nitrogen. The term also includes monocyclic heterocycle fused to an aryl or heteroaryl group in which the point of attachment is on the non-aromatic portion. Examples of “heterocyclyl” include pyrrolidinyl, piperidinyl, piperazinyl, imidazolidinyl, 2,3-dihydrofuro(2,3-b)pyridyl, benzoxazinyl, tetrahydrohydroquinolinyl, tetrahydroisoquinolinyl, dihydroindolyl, and the like. The term also includes partially unsaturated monocyclic rings that are not aromatic, such as 2- or 4-pyridones attached through the nitrogen or N-substituted-(1H,3H)-pyrimidine-2,4-diones (N-substituted uracils).

“Halogen” includes fluorine, chlorine, bromine and iodine.

### Optical Isomers—Diastereomers—Geometric Isomers—Tautomers

Compounds of Formula I contain one or more asymmetric centers and can thus occur as racemates and racemic mixtures, single enantiomers, diastereomeric mixtures and individual diastereomers. The present invention is meant to comprehend all such isomeric forms of the compounds of Formula I.

Some of the compounds described herein contain olefinic double bonds, and unless specified otherwise, are meant to include both E and Z geometric isomers.

Some of the compounds described herein may exist with different points of attachment of hydrogen, referred to as tautomers. Such an example may be a ketone and its enol form known as keto-enol tautomers. The individual tautomers as well as mixture thereof are encompassed with compounds of Formula I.

Compounds of the Formula I may be separated into diastereoisomeric pairs of enantiomers by, for example, fractional crystallization from a suitable solvent, for example MeOH or EtOAc or a mixture thereof. The pair of enantiomers thus obtained may be separated into individual stereoisomers by conventional means, for example by the use of an optically active amine as a resolving agent or on a chiral HPLC column.

Alternatively, any enantiomer of a compound of the general Formula I or Ia may be obtained by stereospecific synthesis using optically pure starting materials or reagents of known configuration.

### Salts

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenedianine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, and tartaric acids.

It will be understood that, as used herein, references to the compounds of Formula I are meant to also include the pharmaceutically acceptable salts.

### Utilities

The ability of the compounds of Formula I to antagonize the actions of VLA-4 integrin makes them useful for preventing or reversing the symptoms, disorders or diseases induced by the binding of VLA-4 to its various ligands. Thus, these antagonists will inhibit cell adhesion processes including cell activation, migration, proliferation and differentiation. Accordingly, another aspect of the present invention provides a method for the treatment (including prevention, alleviation, amelioration or suppression) of diseases or disorders or symptoms mediated by VLA-4 binding and cell adhesion and activation, which comprises administering to a mammal an effective amount of a compound of Formula I. Such diseases, disorders, conditions or symptoms are, for example (1) multiple sclerosis, (2) asthma, (3) allergic rhinitis, (4) allergic conjunctivitis, (5) inflammatory lung diseases, (6) rheumatoid arthritis, (7) septic arthritis, (8) type I diabetes, (9) organ transplantation rejection, (10) restenosis, (11) autologous bone marrow transplantation, (12) inflammatory sequelae of viral infections, (13) myocarditis, (14) inflammatory bowel disease including ulcerative colitis and Crohn's disease, (15) certain types of toxic and immune-based nephritis, (16) contact dermal hypersensitivity, (17) psoriasis, (18) tumor metastasis, (19) atherosclerosis, (20) sickle cell anemia, (21) certain acute leukemias, (22) various melanomas, carcinomas and sarcomas (including multiple myeloma); (23) acute respiratory distress syndrome; (24) uveitis; (25) circulatory shock; (26) hepatitis, and (27) chronic obstructive pulmonary disease. The compounds of the present invention may be useful for the treatment of the above-recited diseases, disorders, conditions or symptoms in mammals other than humans, including, for example, horses, cats, dogs, cows and pigs. The instant compounds may also be useful for the treatment of allergy-related or allergy-induced respiratory conditions in non-human mammals, including the treatment of recurrent airway obstruction, commonly called heaves, in horses.

The utilities of the present compounds in these diseases or disorders may be demonstrated in animal disease models that have been reported in the literature. The following are examples of such animal disease models: i) experimental allergic encephalomyelitis, a model of neuronal demyelination resembling multiple sclerosis (for example, see T. Yednock et al., Nature, 356, 63 (1993) and E. Keszthelyi et al., Neurology, 47, 1053 (1996)); ii) bronchial hyperresponsiveness in sheep and guinea pigs as models for the various phases of asthma (for example, see W. M. Abraham et al., J. Clin. Invest. 93, 776 (1993) and A. A. Y. Milne and P. P. Piper, Eur. J. Pharmacol., 282, 243 (1995)); iii) adjuvant-induced arthritis in rats as a model of inflammatory arthritis (see C. Barbadillo et al., Arthr. Rheuma. (Suppl.), 36 95 (1993) and D. Seiffge, J. Rheumatol., 23, 12 (1996)); iv) adoptive autoimmune diabetes in the NOD mouse (see J. L. Baron et al., J. Clin. Invest., 93, 1700 (1994), A. Jakubowski et al., J. Immunol., 155, 938 (1995), and X. D. Yang et al., Diabetes, 46, 1542 (1997)); v) cardiac allograft survival in mice as a model of organ transplantation (see M. Isobe et al., Transplant. Proc., 26, 867 (1994) and S. Molossi et al., J. Clin Invest., 95, 2601 (1995)); vi) spontaneous chronic colitis in cotton-top tamarins which resembles human ulcerative colitis, a form of inflammatory bowel disease (see D. K. Podolsky et al., J. Clin. Invest., 92, 372 (1993)); vii) contact hypersensitivity models as a model for skin allergic reactions (see T. A. Ferguson and T. S. Kupper, J. Immunol. 150, 1172 (1993) and P. L. Chisholm et al., Eur. J. Immunol., 23, 682 (1993)); viii) acute nephrotoxic nephritis (see M. S. Mulligan et al., J. Clin. Invest., 91, 577 (1993)); ix) tumor metastasis (for examples, see M. Edward, Curr. Opin. Oncol., 7, 185 (1995)); x) experimental autoimmune thyroiditis (see R. W. McMurray et al., Autoimmunity, 23, 9 (1996); xi) ischemic tissue damage following arterial occlusion in rats (see F. Squadrito et al., Eur. J. Pharmacol., 318, 153 (1996)); xii) inhibition of TH2 T-cell cytokine production including IL-4 and IL-5 by VLA-4 antibodies which would attenuate allergic responses (J. Clinical Investigation 100, 3083 (1997); xiii) antibodies to VLA-4 integrin mobilize long term repopulating cells and augment cytokine-induced mobilization in primates and mice (Blood, 90 4779-4788 (1997); xiv) sickle reticulocytes adhere to VCAM-1 (Blood 85 268-274 (1995) and Blood 88 4348-4358 (1996); xv) chemokine stromal cell derived factor 1 modulates VLA-4 integrin mediated multiple myeloma cell adhesion to CS-1/fibronectin and VCAM-1 (Blood, 97, 346-351 2001); xvi) Anti-α4 integrin antibody suppresses the development of multiple myeloma and associated osteoclastic osteolysis (see Y. Mori et al., Blood, 104 2149-2154).

### Dose Ranges

The magnitude of prophylactic or therapeutic dose of a compound of Formula I will, of course, vary with the nature and severity of the condition to be treated, and with the particular compound of Formula I used and its route of administration. The dose will also vary according to the age, weight and response of the individual patient. In general, the daily dose range lie within the range of from about 0.001 mg to about 100 mg per kg body weight of a mammal, preferably 0.01 mg to about 50 mg per kg, and most preferably 0.1 to 10 mg per kg, in single or divided doses. On the other hand, it may be necessary to use dosages outside these limits in some cases.

For use where a composition for intravenous administration is employed, a suitable dosage range is from about 0.01 mg to about 25 mg (preferably from 0.1 mg to about 10 mg) of a compound of Formula I per kg of body weight per day.

In the case where an oral composition is employed, a suitable dosage range is, e.g. from about 0.01 mg to about 100 mg of a compound of Formula I per kg of body weight per day, preferably from about 0.11 mg to about 10 mg per kg.

For use where a composition for sublingual administration is employed, a suitable dosage range is from 0.01 mg to about 25 mg (preferably from 0.1 mg to about 5 mg) of a compound of Formula I per kg of body weight per day.

For the treatment of asthma, a compound of Formula I may be used at a dose of from about 0.1 mg/kg to about 100 mg/kg, preferably from about 1 mg/kg to 10 mg/kg, by oral/inhalation/sublingual/etc. once, twice, three times daily, etc. The dose may be administered as a single daily dose or divided for twice or thrice daily administration.

For the treatment of multiple sclerosis, a compound of Formula I may be used at a dose of from about 0.1 mg/kg to about 100 mg/kg, preferably from about 1 mg/kg to 10 mg/kg, by oral/inhalation/sublingual/etc. once, twice, three times daily, etc. The dose may be administered as a single daily dose or divided for twice or thrice daily administration.

For the treatment of inflammatory bowel disease, a compound of Formula I may be used at a dose of from about 0.1 mg/kg to about 100 mg/kg, preferably from about 1 mg/kg to 10 mg/kg, by oral/inhalation/etc. once, twice, three times daily, etc. The dose may be administered as a single daily dose or divided for twice or thrice daily administration.

For the treatment of rheumatoid arthritis, a compound of Formula I may be used at a dose of from about 0.1 mg/kg to about 100 mg/kg, preferably from about 1 mg/kg to 10 mg/kg, by oral/inhalation/sublingual/etc. once, twice, three times daily, etc. The dose may be administered as a single daily dose or divided for twice or thrice daily administration.

### Pharmaceutical Compositions

Another aspect of the present invention provides pharmaceutical compositions which comprises a compound of Formula I and a pharmaceutically acceptable carrier. The term “composition”, as in pharmaceutical composition, is intended to encompass a product comprising the active ingredient(s), and the inert ingredient(s) (pharmaceutically acceptable excipients) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of Formula I, additional active ingredient(s), and pharmaceutically acceptable excipients.

Any suitable route of administration may be employed for providing a mammal, especially a human with an effective dosage of a compound of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like.

The pharmaceutical compositions of the present invention comprise a compound of Formula I as an active ingredient or a pharmaceutically acceptable salt thereof, and may also contain a pharmaceutically acceptable carrier and optionally other therapeutic ingredients. The term “pharmaceutically acceptable salts” refers to salts prepared from pharmaceutically acceptable non-toxic bases or acids including inorganic bases or acids and organic bases or acids.

The compositions include compositions suitable for oral, sublingual, rectal, topical, parenteral (including subcutaneous, intramuscular, and intravenous), ocular (ophthalmic), pulmonary (aerosol inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

For administration by inhalation, the compounds of the present invention are conveniently delivered in the form of an aerosol spray presentation from pressurized packs or nebulizers. The compounds may also be delivered as powders which may be formulated and the powder composition may be inhaled with the aid of an insufflation powder inhaler device. The preferred delivery systems for inhalation are metered dose inhalation (MDI) aerosol, which may be formulated as a suspension or solution of a compound of Formula I in suitable propellants, such as fluorocarbons or hydrocarbons and dry powder inhalation (DPI) aerosol, which may be formulated as a dry powder of a compound of Formula I with or without additional excipients.

Suitable topical formulations of a compound of formula I include transdermal devices, aerosols, creams, ointments, lotions, dusting powders, and the like.

In practical use, the compounds of Formula I can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, capsules and tablets, with the solid oral preparations being preferred over the liquid preparations. Because of their ease of administration, tablets and capsules represent the most advantageous oral dosage unit form in which case solid pharmaceutical carriers are obviously employed. If desired, tablets may be coated by standard aqueous or non-aqueous techniques.

In addition to the common dosage forms set out above, the compounds of Formula I may also be administered by controlled release means and/or delivery devices such as those described in U.S. Pat. Nos. 3,845,770; 3,916,899; 3,536,809; 3,598,123; 3,630,200 and 4,008,719.

Pharmaceutical compositions of the present invention suitable for oral administration may be presented as discrete units such as capsules, cachets or tablets each containing a predetermined amount of the active ingredient, as a powder or granules or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association the active ingredient with the carrier which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet may be prepared by compression or molding, optionally with one or more accessory ingredients. Compressed tablets may be prepared by compressing in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Desirably, each tablet contains from about 1 mg to about 500 mg of the active ingredient and each cachet or capsule contains from about 1 to about 500 mg of the active ingredient.

The following are examples of representative pharmaceutical dosage forms for the compounds of Formula I:

| Inj. Suspension (I.M.) | mg/mL | Tablet | mg/tab. | Capsule | mg/cap. | Aerosol | Per canister |
|---|---|---|---|---|---|---|---|
| Cmpd of Formula I | 10 | Cmpd of Formula I | 25 | Cmpd of Formula I | 25 | Compound of Formula I | 24 mg |
| Methylcellulose | 5.0 | Microcryst. Cellulose | 415 | Lactose Powder | 573.5 | Lecithin, NF Liq. Conc. | 1.2 mg |
| Tween 80 | 0.5 | Povidone | 14.0 | Magnesium Stearate | 1.5 | Trichlorofluoromethane, NF | 4.025 g |
| Benzyl alcohol | 9.0 | Pregelatinized Starch | 43.5 | | | Dichlorodifluoromethane, NF | 12.15 g |
| Benzalkonium chloride | 1.0 | Magnesium Stearate | 2.5 | | | | |
| Water for injection to a | | | 500 | | 600 | | |
| total volume of 1 mL | | | | | | | |

### Combination Therapy

Compounds of Formula I may be used in combination with other drugs that are used in the treatment/prevention/suppression or amelioration of the diseases or conditions for which compounds of Formula I are useful. Such other drugs may be administered, by a route and in an amount commonly used therefor, contemporaneously or sequentially with a compound of Formula I. When a compound of Formula I is used contemporaneously with one or more other drugs, a pharmaceutical composition containing such other drugs in addition to the compound of Formula I is preferred. Accordingly, the pharmaceutical compositions of the present invention include those that also contain one or more other active ingredients, in addition to a compound of Formula I. Examples of other active ingredients that may be combined with a compound of Formula I, either administered separately or in the same pharmaceutical compositions, include, but are not limited to: (a) other VLA-4 antagonists such as those described in U.S. Pat. No. 5,510,332, WO97/03094, WO97/02289, WO96/40781, WO96/22966, WO96/20216, WO96/01644, WO96/06108, WO95/15973 and WO96/31206, as well as natalizumab; (b) steroids such as beclomethasone, methylprednisolone, betamethasone, prednisone, dexamethasone, and hydrocortisone; (c) immunosuppressants such as cyclosporin, tacrolimus, rapamycin and other FK-506 type immunosuppressants; (d) antihistamines (H1-histamine antagonists) such as bromopheniramine, chlorpheniramine, dexchlorpheniramine, triprolidine, clemastine, diphenhydramine, diphenylpyraline, tripelennamine, hydroxyzine, methdilazine, promethazine, trimeprazine, azatadine, cyproheptadine, antazoline, pheniramine pyrilamine, astemizole, terfenadine, loratadine, cetirizine, fexofenadine, descarboethoxyloratadine, and the like; (e) non-steroidal anti-asthmatics such as β2-agonists (terbutaline, metaproterenol, fenoterol, isoetharine, albuterol, bitolterol, salmeterol and pirbuterol), theophylline, cromolyn sodium, atropine, ipratropium bromide, leukotriene antagonists (zafirlukast, montelukast, pranlukast, iralukast, pobilukast, SKB-106,203), leukotriene biosynthesis inhibitors (zileuton, BAY-1005); (f) non-steroidal antiinflammatory agents (NSAIDs) such as propionic acid derivatives (alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid, and tioxaprofen), acetic acid derivatives (indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin, and zomepirac), fenamic acid derivatives (flufenamic acid, meclofenamic acid, mefenamic acid, niflumic acid and tolfenamic acid), biphenylcarboxylic acid derivatives (diflunisal and flufenisal), oxicamns (isoxicam, piroxicam, sudoxicain and tenoxican), salicylates (acetyl salicylic acid, sulfasalazine) and the pyrazolones (apazone, bezpiperylon, feprazone, mofebutazone, oxyphenbutazone, phenylbutazone); (g) cyclooxygenase-2 (COX-2) inhibitors such as celecoxib, rofecoxib, and parecoxib; (h) inhibitors of phosphodiesterase type IV (PDE-IV); (i) antagonists of the chemokine receptors, especially CCR-1, CCR-2, and CCR-3; (j) cholesterol lowering agents such as HNG-CoA reductase inhibitors (lovastatin, simvastatin, pravastatin, fluvastatin, atorvastatin, and other statins), sequestrants (cholestyramine and colestipol), nicotinic acid, fenofibric acid derivatives (gemfibrozil, clofibrat, fenofibrate and benzafibrate), and probucol; (k) anti-diabetic agents such as insulin, sulfonylureas, biguanides (metformin), α-glucosidase inhibitors (acarbose) and glitazones (troglitazone, pioglitazone, englitazone, MCC-555, BRL49653 and the like); (1) preparations of interferon beta (interferon beta-1a, interferon beta-1b); (m) anticholinergic agents such as muscarinic antagonists (ipratropium and tiatropium); (n) current treatments for multiple sclerosis, including prednisolone, glatiramer, deoxyadenosine, mitoxantrone, methotrexate, and cyclophosphamide; (o) p38 kinase inhibitors; (p) other compounds such as 5-aminosalicylic acid and prodrugs thereof, antimetabolites such as azathioprine and 6-mercaptopurine, and cytotoxic cancer chemotherapeutic agents.

The weight ratio of the compound of the Formula I to the second active ingredient may be varied and will depend upon the effective dose of each ingredient. Generally, an effective dose of each will be used. Thus, for example, when a compound of the Formula I is combined with an NSAID the weight ratio of the compound of the Formula I to the NSAID will generally range from about 1000:1 to about 1:1000, preferably about 200:1 to about 1:200. Combinations of a compound of the Formula I and other active ingredients will generally also be within the aforementioned range, but in each case, an effective dose of each active ingredient should be used.

### Prodrugs

Some of the compounds of the invention are prodrugs which covert in vivo to the active moiety. For example, when R¹ is ethyl, such as in examples 1, 4, 8, 9, 12, 14, 15, 18 and 20, the compounds of the invention covert in vivo to the corresponding acid. Such prodrugs are readily identifiable by one having ordinary skill in the art.

Abbreviations that may be used in the following Schemes and Examples include: 4-DMAP: 4-dimethylaminopyridine; MeCN: acetonitrile; BOC: tert-butoxycarbonyl; BOC—ON: 2-(tert-butoxycarbonyloxyimino)-2-phenylacetonitrile; BOP: benzotriazol-1-yloxy-tris(dimethylamino)-phosphonium hexafluorophosphate; brine: saturated NaCl solution; DIPEA: N,N-diisopropylethylamine; DMF: dimethylformamide; DMSO: dimethylsulfoxide; Et: ethyl; EtOAc: ethyl acetate; EtOH: ethanol; g or gm: gram; h or hr: hours; HATU: O-(7-azabenzotriazol-1-yl)-1,1,3,3-tetramethyluronium hexafluorophosphate; HBTU: O-(benzotriazol-1-yl)-1,1,3,3-tetramethyluronium hexafluorophosphate; HOAc: acetic acid; HOAt: 1-hydroxy-7-azabenzotriazole; HOBt: 1-hydroxybenzotriazole; HPLC: high pressure liquid chromatography; in vacuo: rotoevaporation; Me: methyl; MeOH: methanol; mg: milligram; MHz: megahertz; min: minutes; mL: milliliter; munol: millimole; MS or ms: mass spectrum; MsCl: methanesulfonyl chloride; Ph: phenyl; Ph3P: triphenylphosphine; PyBOP: (benzotriazol-1-yloxy)-tripyrrolidinophosphonium hexafluorophosphate; rt: room temperature; TEA: triethylamine; TFA: trifluoroacetic acid; THF: tetrahydrofuran; DCM: Dichloromethane; NMM: N-methylmorhpholine

### Methods of Synthesis

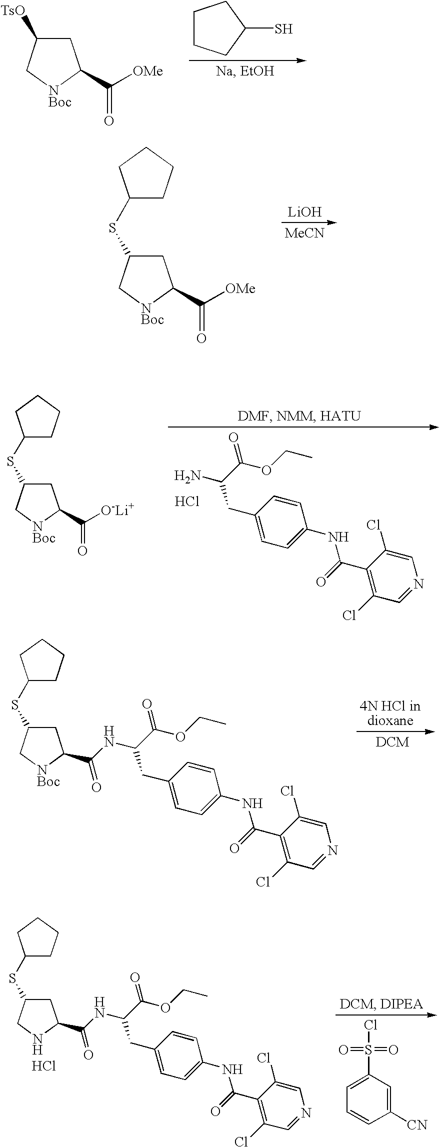

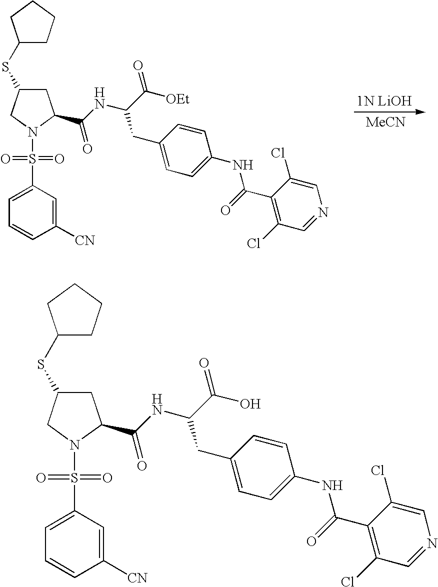

### Biological Evaluation

Compounds of formula I are potent antagonists of VLA-4 with significant and sustained receptor occupancy on VLA-4 bearing cells. The rate of dissociation of a test compound from VLA-4 on Jurkat cells may be determined by the method described in G. Doherty et al., Bioorganic & Medicinal Chemistry Letters, 13, 1891 (2003). Compounds of the present invention had half-lives of dissociation of greater than three hours (t_{1/2}>3 hr) in this assay, demonstrating they are tight binding inhibitors of VLA-4.

VLA-4 receptor occupancy after oral dosing in rats and dogs may be determined by the method described in D. R. Leone et al., J Pharmacol. Exper. Therap., 305, 1150 (2003). Compounds of the present invention are expected to demonstrate sustained and significant receptor occupancy (>50%) after oral dosing.

### EXAMPLES

Compounds of the present invention may be prepared by procedures detailed in the following examples. The examples provided are illustrative of the present invention and are not to be construed as limiting its scope in any manner:

### Example 1

### Ethyl (4R)-1-[(3-cyanophenyl)sulfonyl]-4-(cyclopentylthio)-L-prolyl-4-[(3,5-dichloroisonicotinoyl)amino]-L-phenylalaninate

### Step 1: 1-tert-Butyl 2-methyl (2S,4R)-4-(cyclopentylthio)pyrrolidine-1,2-dicarboxylate

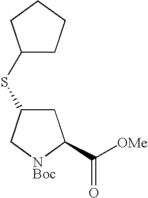

To a solution of cyclopentanethiol (1 mL, 9.30 mmol) in absolute ethanol (9 μL) was added sodium metal (215 mg, 9.35 mmol) and stirred until complete dissolution of the metal. 1-tert-butyl 2-methyl (2S,4S)-4-{[(4-methylphenyl)sulfonyl]oxy}pyrrolidine-1,2-dicarboxylate (1.26 g, 3.15 mmol) was then added in one portion and the reaction mixture maintained at ambient temperature for 12 hr. The solution was diluted with DCM, washed with water, the aqueous layer extracted with DCM and the combined organic phases dried (MgSO₄), filtered and evaporated in vacuo. The residue was purified on silica gel, eluting with a gradient of 0 to 30% acetone in hexanes to afford the title compound as a colorless solid. ¹H NMR (rotamers-CDCl₃, 500 MHz) δ 4.27 (dd, 1H), 4.17 (m, 2H), 3.90 (ddd, 1H), 3.43 (m, 1H), 3.20-3.35 (m, 1H), 3.08 (m, 1H), 2.26 (m, 1H), 2.15 (m, 1H), 2.00 (m, 2H), 1.73 (m, 2H), 1.40-1.62 (m's, 12H), 1.26 (t, 3H).

### Step 2: (4R)-1-(tert-butoxycarbonyl)-4-(cyclopentylthio)-L-proline lithium salt

To a solution of 1-tert-butyl 2-methyl (2S,4R)-4-(cyclopentylthio)pyrrolidine-1,2-dicarboxylate (554 mg, 1.61 mmol) in MeCN (5 mL) was added 1N LiOH soln. (1.7 mL, 1.70 mmol) and the mixture stirred for 12 hr. Evaporation of the solvent in vacuo afforded the title lithium salt which was used without further purification. LCMS (ESI) 314 (M−H)⁻.

### Step 3: Ethyl (4R)-1-(tert-butoxycarbonyl)-4-(cyclopentylthio)-L-prolyl-4-[(3,5-dichloroisonicotinoyl)-amino]-L-phenylalaninate

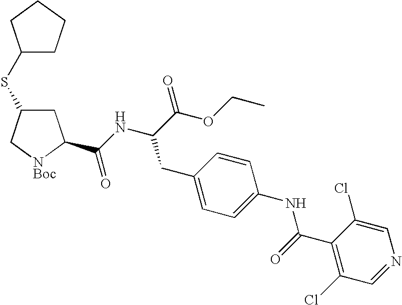

To a solution of (4R)-1-(tert-butoxycarbonyl)-4-(cyclopentylthio)-L-proline lithium salt (556 mg, 1.61 mmol) in DMF (12 mL) was added N-methylmorpholine (0.53 mL, 4.83 mmol) and HATU (672 mg, 1.77 mmol) and the mixture was stirred at ambient temperature for 45 min. Ethyl 4-[(3,5-dichloroisonicotinoyl)amino]-L-phenylalaninate HCl salt (708 mg, 1.69 mmol) was added in one portion and after 30 min the solution was diluted with EtOAc, washed with water and the organic phase dried (MgSO₄), filtered and evaporated in vacuo. The residue was further purified on silica gel eluting with a gradient of 0 to 60% EtOAc in hexanes to afford the title amide as a colorless foam. LCMS (ESI) 701.4 (M+H)⁺.

### Step 4: (4R)-4-(cyclopenthythio)-L-prolyl-4-[(3,5-dichloroisonicotinoyl)amino]-L-phenylalanine

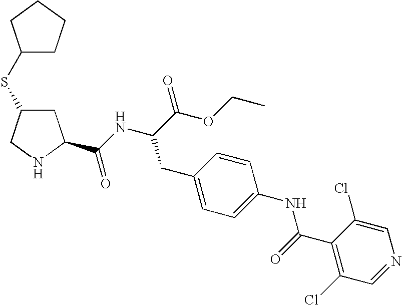

Ethyl (4R)-1-(tert-butoxycarbonyl)-4-(cyclopentylthio)-L-prolyl-4-[(3,5-dichloro-isonicotinoyl)amino]-L-phenylalaninate (645 mg, 0.95 mmol) was dissolved in DCM (6 mL) and 4N HCl in dioxane (2.0 mL) added. The resulting suspension was vigorously stirred until LCMS analysis indicated complete reaction after which the solvent was removed under vacuum to afford the title compound isolated as a yellow mono HCl salt. LCMS (ESI) 581.3 (M+H)⁺.

### Step 5: Ethyl (4R)-[(3-cyanophenyl)sulfonyl]-4-(cyclopentylthio)-L-prolyl-4-[(3,5-dichloro-isonicotinoyl)amino]-L-phenylalaninate

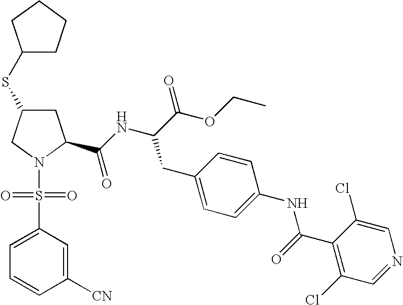

To a suspension of (4R)-4-(cyclopentylthio)-L-prolyl-4-[(3,5-dichloroisonicotinoyl)-amino]-L-phenylalanine HCl salt (585 mg, 0.95 mmol) in DCM was added diisopropylethylamine (0.5 mL, 2.85 mmol) followed by 3-cyanobenzenesulfonyl chloride (230 mg, 1.14 mmol). After 10 min, the solution was diluted with DCM, washed with water, the organic phase dried (MgSO₄), filtered and evaporated in vacuo. The residue was purified using reverse phase HPLC to afford the title compound as an off white foam. ¹H NMR (CDCl₃, 500 MHz) δ 8.61 (br s, 2H), 8.21 (m, 1H), 8.05 (d, 1H), 7.91 (dd, 1H), 7.75 (s, 1H), 7.70 (m, 1H), 7.56 (d, 1H), 7.22 (d, 2H), 7.17 (d, 1H), 6.75 (br s, 2H), 4.85 (m, 1H), 4.26 (m, 2H), 4.20 (dd, 1H), 3.76 (dd, 1H), 3.20-3.29 (m, 2H), 3.13 (dd, 1H), 3.07 (dd, 1H), 2.94 (m, 1H), 2.30 (m, 1H), 1.91 (m, 2H), 1.65-1.77 (m, 3H), 1.55 (m, 2H), 1.35 (m, 2H), 1.32 (t, 3H); LCMS (ESI) 744.3 (M+H)⁺.

### Example 2

### (4R)-1-[(3-Cyanophenyl)sulfonyl]-4-(cyclopentylthio)-L-prolyl-4-[(3,5-dichloroisonicotinoyl)amino]-L-phenylalanine

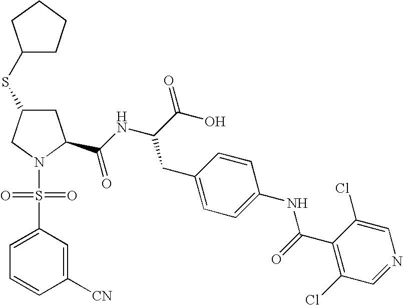

Ethyl (4R)-1-[(3-cyanophenyl)sulfonyl]-4-(cyclopentylthio)-L-prolyl-4-[(3,5-dichloro-isonicotinoyl)amino]-L-phenylalaninate from EXAMPLE 1 (300 mg, 0.403 mmol) was dissolved in MeCN (2 mL) and 1N LiOH soln. (1 mL, 1.00 mmol) was added. The reaction was carefully monitored by LCMS and upon completion (˜30 min) was quenched with acetic acid and evaporated to dryness. The residue was purified using reverse phase HPLC to afford the carboxylic acid as a colorless foam. ¹H NMR (d₃-MeOD, 500 MHz) δ 8.62 (s, 2H), 8.19 (s, 1H), 8.01 (m, 2H), 7.74 (app t, 1H), 7.62 (d, 2H), 7.34 (d, 2H), 4.71 (m, 1H), 4.30 (dd, 1H), 3.80 (dd, 1H), 3.21-3.38 (m's, 2H), 3.07 (dd, 1H), 2.96 (m, 1H), 2.16 (m, 1H), 1.90 (m, 3H), 1.67 (m, 2H), 1.54 (m, 2H), 1.36 (m, 1H), 1.27 (m, 1H); LCMS (ESI) 714.4 (M−H)⁻, 716.3 (M+H)⁺.

The following examples of the invention were made following synthetic methods analogous to the examples and methods of synthesis described above.

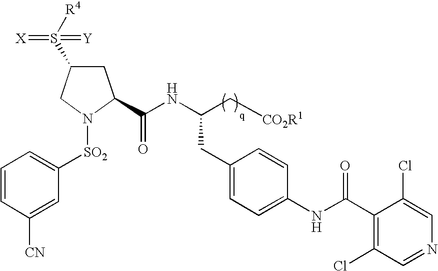

| Ex. | X | Y | q | R¹ | R⁴ | LCMS (ESI) |
|---|---|---|---|---|---|---|
| 3 | oxygen | not | 0 | hydrogen | cyclopentyl | 732.3 |
| | | present | | | | |
| 4 | not | not | 0 | ethyl | tert-butyl | 732.5 |
| | present | present | | | | |
| 5 | not | not | 0 | hydrogen | tert-butyl | 704.3 |
| | present | present | | | | |
| 6 | oxygen | oxygen | 0 | hydrogen | cyclopentyl | 748.3 |
| 7 | not | not | 0 | hydrogen | cyclohexyl | 730.4 |
| | present | present | | | | |
| 8 | not | not | 0 | ethyl | cyclohexyl | 758.4 |
| | present | present | | | | |
| 9 | not | not | 0 | ethyl | phenyl | 752.4 |
| | present | present | | | | |
| 10 | not | not | 0 | hydrogen | phenyl | 724.4 |
| | present | present | | | | |
| 11 | not | not | 0 | hydrogen | 1,2- | 718.6 |
| | present | present | | | dimethylpropyl | |
| 12 | not | not | 0 | ethyl | 1,2- | 746.2 |
| | present | present | | | dimethylpropyl | |
| 13 | not | not | 1 | hydrogen | cyclopentyl | 718.5 |
| | present | present | | | | |
| 14 | not | not | 1 | ethyl | cyclopentyl | 744.2 |
| | present | present | | | | |

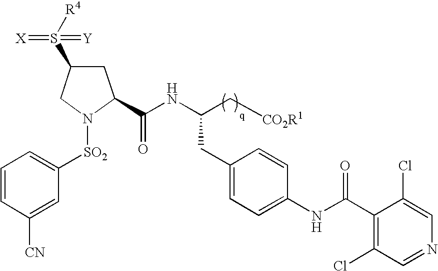

| Ex. | X | Y | q | R¹ | R⁴ | LCMS(ESI) |
|---|---|---|---|---|---|---|
| 15 | not | not | 0 | ethyl | cyclopentyl | 742.5 (M − H)⁻ |
| | present | present | | | | |
| 16 | not | not | 0 | hydrogen | cyclopentyl | 716.4 |
| | present | present | | | | |
| 17 | oxygen | not | 0 | hydrogen | cyclopentyl | 730.6 (M − H)⁻ |
| | | present | | | | |
| 18 | oxygen | not | 0 | ethyl | cyclopentyl | 758.1 (M − H)⁻ |
| | | present | | | | |
| 19 | oxygen | oxygen | 0 | hydrogen | cyclopentyl | 748.5 |
| 20 | oxygen | oxygen | 0 | ethyl | cyclopentyl | 776.4 |

What is claimed is:

1. A compound of formula I: 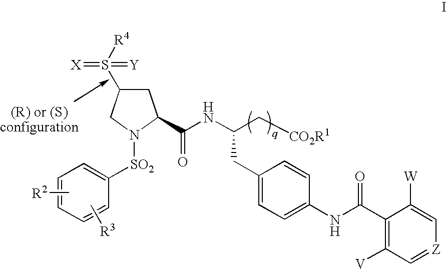
or a pharmaceutically acceptable salt thereof, wherein:
q is 0 or 1;
V and W are independently chosen from (1) C₁₋₃ alkyl, (2) halogen, and (3) C₁₋₃alkoxy;
X and Y may independently be oxygen or not present;
Z is N or N⁺O⁻;
R¹ is selected from (1) hydrogen, (2) C₁₋₁₀alkyl, (3) —(C₁₋₁₀alkyl)-aryl, (4) —(C₁₋₁₀alkyl)-O—C₁₋₁₀alkyl, (5) —(C₁₋₁₀ alkyl)-OC(O)—C₁₋₁₀alkyl, (6) —(C₁₋₁₀alkyl)-OC(O)-aryl, (7) —(C₁₋₁₀ alkyl)-OC(O)O—C₁₋₁₀alkyl, and (8) —(C₁₋₁₀ alkyl)-N⁺(C₁₋₃ alkyl)₃; wherein alkyl is optionally substituted with one to three substituents independently selected from R^{a}, and aryl is optionally substituted with one to three substituents independently selected from R^{b};
R² and R³ are independently selected from H, —SO₂—C₁₋₃alkyl, CN, CF₃, OCF₃, and halogen;
R⁴ is selected from the group consisting of: C₁₋₁₀alkyl, C₂₋₁₀alkenyl, C₂₋₁₀alkynyl, Cy and Cy-C₁₋₁₀alkyl, wherein alkyl, alkenyl, alkynyl and Cy are optionally substituted with one to four substituents independently selected from R^{c};
R^{a} is selected from (1) —OR^{d}, (2) —NR^{d}S(O)ₘR^{e}, (3) —NO₂, (4) halogen, (5) —S(O)ₘR^{d}, (6) —SR^{d}, (7) —S(O)₂OR^{d}, (8) —S(O)ₘNR^{d}R^{e}, (9) —NR^{d}R^{e}, (10) —O(CR^{f}R^{g})ₙNR^{d}R^{e}, (11) —C(O)R^{d}, (12) —CO₂R^{d}, (13) —CO₂(CR^{f}R^{g})ₙCONR^{d}R^{e}, (14) —OC(O)R^{d}, (15) —CN, (16) —C(O)NR^{d}R^{e}, (17) —NR^{d}C(O)R^{e}, (18) —OC(O)NR^{d}R^{e}, (19) —NR^{d}C(O)OR^{e}, (20) —NR^{d}C(O)NR^{d}R^{e}, (21) —CR^{d}(N—OR^{e}), (22) CF₃, (23) —OCF₃, (24) C₃₋₈cycloalkyl, and (25) heterocyclyl; wherein cycloalkyl and heterocyclyl are optionally substituted with one to three groups independently selected from R^{c};
R^{b} is selected from (1) a group selected from R^{a}, (2) C₁₋₁₀ alkyl, (3) C₂₋₁₀ alkenyl (4) C₂₋₁₀ alkynyl, (5) aryl, and (6) —(C₁₋₁₀alkyl)-aryl, wherein alkyl, alkenyl, alkynyl, and aryl are optionally substituted with one to three substituents selected from a group independently selected from R^{c};
R^{c} is (1) halogen, (2) amino, (3) carboxy, (4) C₁₋₄alkyl, (5) C₁₋₄alkoxy, (6) aryl, (7) —(C₁₋₄alkyl)-aryl, (8) hydroxy, (9) CF₃, (10) OC(O)C₁₋₄alkyl, (11) —CN, and (12) —SO₂C₁₋₁₀alkyl;
R^{d} and R^{e} are independently selected from hydrogen, C₁₋₁₀alkyl, C₂₋₁₀alkenyl, C₂₋₁₀alkynyl, Cy and Cy-C₁₋₁₀alkyl, wherein alkyl, alkenyl, alkynyl and Cy are optionally substituted with one to four substituents independently selected from R^{c}; or
R^{d} and R^{e} together with the atom(s) to which they are attached form a heterocyclic ring of 4 to 7 members containing 0-2 additional heteroatoms independently selected from O, S and N—R^{h}; wherein said ring is optionally substituted with one to four substituents independently selected from R^{c};
R^{f} and R^{g} are independently selected from hydrogen, C₁₋₁₀ alkyl, Cy and Cy-C₁₋₁₀ alkyl; or
R^{f} and R^{g} together with the carbon to which they are attached form a ring of 5 to 7 members containing 0-2 heteroatoms independently selected from oxygen, sulfur and nitrogen;
R^{h} is selected from R^{f} and —C(O)R^{f};
Cy is selected from cycloalkyl, heterocyclyl, aryl, and heteroaryl;
each m is independently 0, 1 or 2; and
each n is independently 1, 2, 3, or 4.

2. The compound according to claim 1 wherein one of V and W is halogen and the other is selected from halogen, C₁₋₃alkyl and C₁₋₃alkoxy.

3. The compound according to claim 2 wherein one of V and W is chloro and the other is chloro or methoxy.

4. The compound according to claim 3 wherein V and W are each chloro.

5. The compound according to claim 1 wherein R¹ is selected from the group consisting of: hydrogen, C₁₋₄alkyl, —(C₁₋₄alkyl)OC(O)—C₁₋₄alkyl, and —(C₁₋₄alkyl)OC(O)—C₁₋₄alkyl.

6. The compound according to claim 1 wherein R¹ is hydrogen.

7. The compound according to claim 1 wherein R¹ is C₁₋₄alkyl.

8. The compound according to claim 1 wherein R² is hydrogen and R³ is CN.

9. The compound according to claim 1 wherein R⁴ is selected from the group consisting of: C₁₋₆alkyl, cycloalkyl and aryl.

10. The compound according to claim 1 of formula Ia: 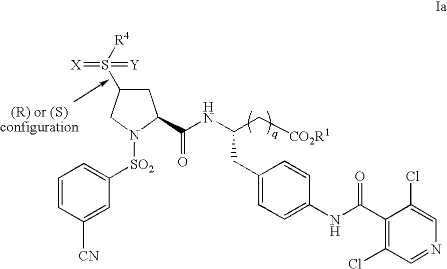
or a pharmaceutically acceptable salt thereof, wherein
q is 0 or 1;
X and Y may independently be oxygen or not present;
R¹ is selected from hydrogen and ethyl; and
R⁴ is selected from C₁₋₆alkyl, cyclopentyl, cyclohexyl and phenyl.

11. The compound according to claim 10 wherein q is 0.

12. The compound according to claim 10 wherein q is 1.

13. The compound according to claim 10 wherein X and Y are not present.

14. The compound according to claim 10 wherein X is oxygen and Y is not present.

15. The compound according to claim 10 wherein X and Y are oxygen.

16. A compound according to claim 10 selected from one of the following tables:

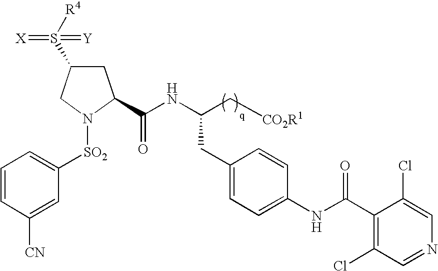

| X | Y | q | R¹ | R⁴ |
|---|---|---|---|---|
| not present | not present | 0 | ethyl | cyclopentyl |
| not present | not present | 0 | hydrogen | cyclopentyl |
| oxygen | not present | 0 | hydrogen | cyclopentyl |
| not present | not present | 0 | ethyl | tert-butyl |
| not present | not present | 0 | hydrogen | tert-butyl |
| oxygen | oxygen | 0 | hydrogen | cyclopentyl |
| not present | not present | 0 | hydrogen | cyclohexyl |
| not present | not present | 0 | ethyl | cyclohexyl |
| not present | not present | 0 | ethyl | phenyl |
| not present | not present | 0 | hydrogen | phenyl |
| not present | not present | 0 | hydrogen | 1,2-dimethylpropyl |
| not present | not present | 0 | ethyl | 1,2-dimethylpropyl |
| not present | not present | 1 | hydrogen | cyclopentyl |
| not present | not present | 1 | ethyl | cyclopentyl |

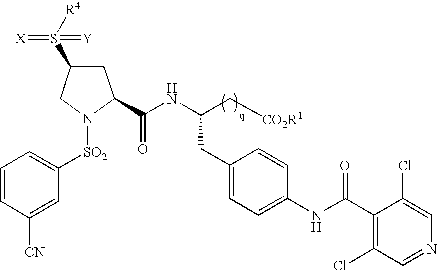
| X | Y | q | R¹ | R⁴ |
|---|---|---|---|---|
| not present | not present | 0 | ethyl | cyclopentyl |
| not present | not present | 0 | hydrogen | cyclopentyl |
| oxygen | not present | 0 | hydrogen | cyclopentyl |
| oxygen | not present | 0 | ethyl | cyclopentyl |
| oxygen | oxygen | 0 | hydrogen | cyclopentyl |
| oxygen | oxygen | 0 | ethyl | cyclopentyl |

or a pharmaceutically acceptable salt of any of the above.

17. A pharmaceutical composition comprising a therapeutically effective amount of a compound of claim 1 or a pharmaceutically acceptable salt thereof and a pharmaceutically acceptable carrier.

18. Use of a compound of claim 1 or a pharmaceutically acceptable salt thereof for the manufacture of a medicament for the treatment or prevention of diseases mediated by cell adhesion.

19. The use of claim 18 wherein said disease is selected from asthma, multiple sclerosis, inflammatory bowel disease, chronic obstructory pulmonary disease, sickle cell anemia, leukemia, multiple myeloma, and rheumatoid arthritis.

20. A method for preventing the action of VLA-4 in a mammal which comprises administering to said mammal a therapeutically effective amount of a compound according to claim 1.
